(19) United States
(12) Patent Application Publication
(10) Pub. No.: US 2025/0387271 A1
(43) Pub. Date: Dec. 25, 2025

(54) THERMORESPONSIVE WOUND DRESSING

(71) Applicant: Baden-Württemberg Stiftung gGmbH, Stuttgar (DE)

(72) Inventors: Benjamin STOLZ, Freiburg (DE); Rolf MÜLHAUPT, Freiburg (DE); Thorsten STEINBERG, Mannheim (DE)

(21) Appl. No.: 18/727,560

(22) PCT Filed: Nov. 8, 2022
(86) PCT No.: PCT/EP2022/081113

§ 371 Date: Mar. 11, 2025

(30) Foreign Application Priority Data
Jan. 11, 2022 (DE) 10 2022 100 519.4

(51) Int. Cl. A61F 13/0246; A61L 15/58

(52) U.S. Cl. CPC A61F 13/0253; A61L 15/58

(57) ABSTRACT

The present invention relates to thermoresponsive wound dressings with thermally switchable adhesion/detachment, comprising:
a carrier material, and
an adhesive composition applied to a surface of the carrier material, the adhesive composition comprising:
at least one vinyl alcohol/copolymer with the following formula (I):

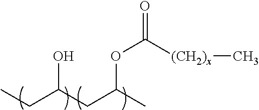

where x is an integer from 10 to 18 independently of one another with respect to each fatty acid unit,
where the copolymer according to formula (I) is present at body temperature as a melt that adheres to wounds and skin, and upon cooling to a temperature above the freezing point of water, the crystallization of the copolymer according to formula (I) causes the wound dressing to detach,
and a use of said thermoresponsive wound dressing for wound healing or for protection against skin injuries and skin disorders.

The present invention relates to thermoresponsive wound dressings with thermally switchable adhesion/detachment and the use of these thermoresponsive wound dressings for wound healing and for protection against skin injuries and skin disorders.

Even today, the majority of self-adhesive wound dressings used are based on the Hansaplast® or Leukoplast® system developed over 100 years ago, in particular on the basis of zinc oxide rubber adhesive. In order to improve the painful removal of these wound dressings and to minimize the adhesive residues left on the wound, research has been carried out for over 30 years on alternative adhesive materials based on the “Bond/Debond-on-Command” principle (cf. U.S. Pat. No. 5,156,911).

The functional principle of these materials is that they adhere reliably at body temperature and detach by cooling or heating in a controlled manner. This temperature-induced transition from adhesion to detachment can currently be implemented with two strategies. On the one hand, the switchable stickiness can be achieved with a crystallization process. On the other hand, the adhesive material can exhibit the desired behavior by thermoreversible switching between hydrophilic and hydrophobic character.

The first strategy is based on thermoresponsive polymers with crystallizable side chains, which are solid at room temperature and melted at body temperature and only have adhesion in the melted state. When cooled, these side chains crystallize, which leads to a loss of adhesion to the skin. The crystallization causes a physical cross-linking of the material, which greatly inhibits adhesion to the skin and at the same time increases the cohesion of the material. In this way, the corresponding wound dressing can be removed in one piece without leaving any adhesive residue and without causing pain. At the same time, there is a reduction in volume, which reduces the contact area between the wound dressing and the skin and supports the removal of the corresponding wound dressing. Thermoresponsive polymers that were not suitable as pressure-sensitive adhesives for skin applications themselves were added as additives to other pressure-sensitive adhesives in skin applications (cf. U.S. Pat. No. 5,156,911 and R. A. Chivers, International Journal of Adhesion and Adhesives 2001, 21, 381-388). The crystallization temperature and crystallinity of vinyl ester polymers can be adjusted via the side chain length (cf. D. Heinze et al., Thermochimica Acta 2016, 637, 143-153)

In this context, U.S. Pat. No. 5,156,911 describes the free radical copolymerization of hexadecyl acrylate with ethyl acrylate in a mass ratio of 5:1, which yields statistical vinyl ester copolymers with a melting temperature of 34° C. that adhere at human body temperature and can be easily removed from the skin by cooling. Here, the ethyl chains of ethyl acrylate are too short to crystallize, whereas the hexadecyl chains of the hexadecyl acrylate initiate side chain crystallization upon cooling. This principle can be transferred to other acrylate-based systems. For example, the free radical copolymerization of pentadecyl acrylate and acrylic acid in a mass ratio of 19:1 or of hexadecyl acrylate, isodecyl acrylate and acrylic acid in a mass ratio of 16:3:1 each leads to statistical copolymers with similar properties.

The second strategy for obtaining thermoreversible stickiness in wound dressings is based on a change between hydrophilicity and hydrophobicity due to a lower critical solution temperature (LCST) or an upper critical solution temperature (UCST). In an LCST-induced change, intermolecular hydrogen bonds between polymer chains and water molecules are thermodynamically favored below the LOST and hydrophilic behavior dominates. By increasing the temperature above the LOST, intramolecular interactions are favored, which makes the polymer hydrophobic. In a UCST-based change between hydrophilicity and hydrophobicity, the behavior is reversed, which leads to hydrophobization by cooling below the UCST (cf. H. Yamauchi et al., The Journal of Physical Chemistry. B 2007, 111, 12964-12968 and A. Gandhi et al., Asian Journal of Pharmaceutical Sciences 2015, 10, 99-107). Examples of materials with LCST known from the literature include hydrogels made of cross-linked polyvinyl alcohol (PVA) as a carrier material with cross-linked poly-N-isopropylacrylamide (PNIPAm). Here, PNIPAm has an LCST which, depending on the ratio of PNIPAm to PVA, is between 3° and 37° C. However, this hydrogel is not suitable for use as an adhesive for a wound dressing, as the LOST must be above human body temperature (cf. J.-T. Zhang, et al., Colloid and Polymer Science 2003, 281, 580-583 and A. C. Wenceslau et al., Materials Science and Engineering: C 2012, 32, 1259-1265). An LCST-based system, which is already used in wound dressings, consists of polypropylene (PP), chitosan and PNIPAm. Acrylic acid is first grafted onto the corresponding PP and then chitosan and PNIPAm are bound using a carbodiimide as a coupling reagent (cf. J.-P. Chen et al., Applied Surface Science 2012, 262, 95-101).

Q. Wang et al. have already shown a combination of cross-linked polyacrylic acid (PAAc) with cross-linked polyacrylamide (PAAm) as an example of suitable UCST-based hydrogels. The UCST of the hydrogel is already at 35° C., i.e. at the upper limit of the required range. Here, cooling leads to phase separation and the hydrogel detaches from the aqueous wound bed. It was also shown that β-cyclodextrin can be grafted onto the PAAc network, which can serve to release additional drugs (Q. Wang et al., Journal of Applied Polymer Science 2009, 111, 1417-1425).

In addition to the temperature-induced “bond/debond-on-command”, it is possible to control the adhesion and detachment of adhesive materials by exposure to UV light. Upon irradiation, a covalent network with significantly reduced adhesion is formed through a photochemical reaction. However, these systems are not suitable as adhesive materials for wound dressings, as the use of photosensitive components such as epoxy acrylates, photoinitiators or transition metal complexes in contact with the wound is to be considered critical (W. Zhang et al., Journal of Applied Polymer Science 2018, 135, 46435).

It can thus be stated that various adhesive materials based on the “bond/debond-on-command” principle are already prior art. However, it is also clear that there is still a need for improvement for such adhesive materials with regard to (i) adhesive residues left on the patient when they are removed painlessly and (ii) biocompatibility. In particular during inpatient wound treatment of patients, adhesive residues can lead to complications with the corresponding skin injury and must be laboriously removed by nursing staff.

Accordingly, it is an object of the present invention to provide thermoresponsive wound dressings comprising corresponding adhesive compositions, which ensure painless removal for the patient, wherein the residues of the adhesive composition remaining on the patient are minimized, and which also have excellent biocompatibility.

The object described above is achieved by the embodiments of the present invention characterized in the claims.

In particular, according to the invention, thermoresponsive wound dressings with thermally switchable adhesion/detachment are provided, comprising:
a carrier material, and
an adhesive composition applied to a surface of the carrier material, the adhesive composition comprising:
at least one vinyl alcohol/copolymer with the following formula (I):

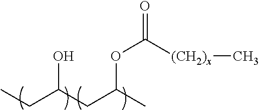

where x is an integer from 10 to 18 independently of one another with respect to each fatty acid unit,
where the copolymer according to formula (I) is present at body temperature as a melt that adheres to wounds and skin, and upon cooling to a temperature above the freezing point of water, the crystallization of the copolymer according to formula (I) causes the wound dressing to detach.

The use of the above-defined adhesive composition advantageously leads to the thermoresponsive wound dressings according to the invention being able to be removed from the patient after use without pain and with minimal residues, and the thermoresponsive wound dressings according to the invention also have excellent biocompatibility. In this context, it was surprisingly discovered that fatty acid esters of polyvinyl alcohol are characterized by thermally switchable adhesion/detachment and residue-free detachment.

The term “thermoresponsive” according to the present invention means that the physical properties of the at least one copolymer represented by the formula (I) change drastically and discontinuously with temperature, in particular exhibit a thermally switchable phase transition between crystalline and amorphous below body temperature and above the freezing point of water.

In particular, if the at least one copolymer with the formula (I) has a melting temperature (Tₘ) of 20 to 35° C. and a melting enthalpy (ΔHₘ) of 20 J/g or more, the copolymer according to formula (I) is present at body temperature as a melt that adheres well to wounds and skin. If a copolymer according to formula (I) with such a melting temperature and such a melting enthalpy is present, the crystallization of the copolymer according to formula (I) causes the wound dressing to detach when cooled to a temperature above the freezing point of water.

The above-defined at least one copolymer with the formula (I) preferably has a melting temperature (Tₘ) of 22 to 33° C., more preferably 24 to 31° C., particularly preferably 26 to 29° C. For example, the at least one copolymer with the formula (I) can have a melting temperature (Tₘ) of 20 to 33° C., 20 to 31° C., 20 to 29° C., 22 to 35° C., 22 to 31° C., 22 to 29° C., 24 to 35° C., 24 to 33° C., 24 to 29° C., 26 to 35° C., 26 to 33° C. or 26 to 31° C.

Furthermore, the above-defined at least one copolymer with the formula (I) preferably has a melting enthalpy (ΔHₘ) of 25 J/g or more, more preferably 30 J/g or more, particularly preferably 35 J/g or more.

The melting temperature (Tₘ) and the melting enthalpy (ΔHₘ) of the above-defined at least one copolymer with the formula (I) can be determined, for example, by means of DSC (differential scanning calorimetry) at a heating rate of 10 K/min.

It should be noted that the PVA/fatty acid ester copolymers used according to the invention are not accessible with the method described in U.S. Pat. No. 5,156,911. For example, by esterifying polyvinyl alcohol with fatty acids via side chain crystallization, the melting and crystallization temperature can be specifically set below body temperature. Polyvinyl alcohol is biocompatible and well tolerated by the skin, but only exhibits main chain crystallization at temperatures well above body temperature. While the hydrolysis of the PVA/fatty acid ester copolymers used according to the invention advantageously produces skin-compatible polyvinyl alcohol, the hydrolysis of the vinyl ester copolymers or (meth)acrylate copolymers described in U.S. Pat. No. 5,156,911 produces skin-irritating poly(meth)acrylic acid. In addition, the molecular architectures of the PVA/fatty acid ester copolymers used according to the invention can be used to achieve residue-free removal.

According to the invention, the above-mentioned adhesive composition for the thermoresponsive wound dressings according to the invention is not further restricted, provided that it comprises at least one copolymer with the formula (I) shown above. The copolymer can be a statistical copolymer, a gradient polymer or a block polymer, virtually constructed from vinyl alcohol units and vinyl fatty acid ester units. Preferably, particularly in view of the simpler production by esterification of polyvinyl alcohol with fatty acids, the copolymer according to formula (I) is a statistical copolymer. Here, the adhesive composition can comprise a statistical copolymer or two or more statistical copolymers with the above-mentioned formula (I), wherein the two or more statistical copolymers are different from one another.

Preferably, the molar ratio of vinyl alcohol units to vinyl fatty acid ester units is 0.02 to 0.80, preferably 0.03 to 0.70, more preferably 0.04 to 0.60, particularly preferably 0.05 to 0.50. For example, the molar ratio of vinyl alcohol units to vinyl fatty acid ester units can be 0.02 to 0.70, 0.02 to 0.60, 0.02 to 0.50, 0.03 to 0.80, 0.03 to 0.60, 0.03 to 0.50, 0.04 to 0.80, 0.04 to 0.70, 0.04 to 0.50, 0.05 to 0.80, 0.05 to 0.70, or 0.05 to 0.60.

In a particularly preferred embodiment of the present invention, x is 12 and the molar ratio of vinyl alcohol units to vinyl fatty acid ester units is 0.05 to 0.50.

The ratio of vinyl alcohol units to vinyl fatty acid ester units of the at least one, preferably statistical, copolymer with the formula (I) shown above can be determined by means of ¹H-NMR spectroscopy in CDCl₃ as a solvent, for example. The fact that it is preferably a statistical copolymer of the formula (I) shown above, rather than, for example, two homopolymers of the respective monomers used, can be determined by means of 2D-DOSY-NMR spectroscopy in CDCl₃, for example. If the statistical copolymerization of the respective monomers is successful, all signals have the same diffusion coefficient in the respective deuterated solvent used, for example CDCl₃.

The at least one copolymer with the formula (I) shown above can optionally also comprise one or more further units derived from monomers selected from ethylene and vinyl acetate, in proportions as long as the thermoresponsive properties of the copolymer are not adversely affected.

In a further embodiment, the at least one copolymer has a number-average molecular weight (Mₙ) of 1000 to 100,000 g/mol, preferably 3000 to 90,000 g/mol, more preferably 5000 to 80,000 g/mol, particularly preferably 10,000 to 70,000 g/mol. For example, the at least one copolymer according to formula (I) can have a Mₙ of 1000 to 90000 g/mol, 1000 to 80000 g/mol, 1000 to 70000 g/mol, 3000 to 100000 g/mol, 3000 to 80000 g/mol, 3000 to 70000 g/mol, 5000 to 100000 g/mol, 5000 to 90000 g/mol, 5000 to 70000 g/mol, 10000 to 100000 g/mol, 10000 to 90000 g/mol or 10000 to 80000 g/mol.

The polydispersity index (PDI) of the at least one copolymer is not further restricted and can be, for example, 1.00 to 3.00, 1.00 to 2.50, 1.00 to 2.00 or 1.00 to 1.50. The PDI corresponds to the width of the frequency distribution of individual molar masses.

Various methodologies for determining the Mₙ and PDI of a copolymer are known to the person skilled in the art, such as MALDI-TOF mass spectrometry, ESI-ToF mass spectrometry, gel permeation chromatography (GPC, SEC), asymmetric flow field flow fractionation (AF4), vapor pressure osmometry and ¹H-NMR spectroscopy. The Mₙ and PDI of the at least one statistical copolymer can be determined by gel permeation chromatography (GPC), for example.

In a further embodiment, the adhesive composition comprises the at least one copolymer in an amount of 40 to 100 wt. %, preferably 50 to 99 wt. %, more preferably 60 to 95 wt. %, particularly preferably 70 to 90 wt. %. For example, the adhesive composition can comprise the at least one copolymer in an amount of 40 to 99 wt. %, 40 to 95 wt. %, 40 to 90 wt. %, 50 to 100 wt. %, 50 to 95 wt. %, 50 to 90 wt. %, 60 to 100 wt. %, 60 to 99 wt. %, 60 to 90 wt. %, 70 to 100 wt. %, 70 to 99 wt. % or 70 to 95 wt. %.

In a preferred embodiment of the above-defined adhesive composition, below the melting temperature (Tₘ) of the at least one copolymer, the side chain crystallinity of the at least one copolymer is in the form of an “end-on” crystal structure, particularly preferably exclusively in the form of an “end-on” crystal structure.

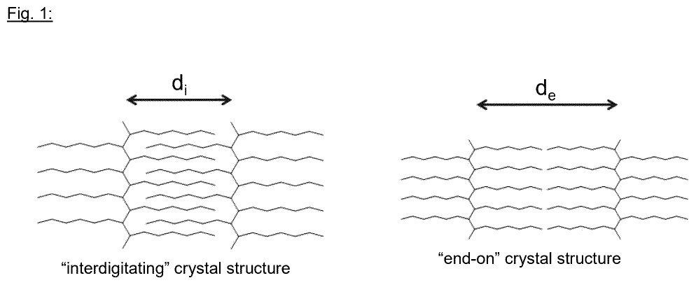

In the case of side chain crystallization of a polymer, there are two different crystallization mechanisms by which the side chains can accumulate to form crystallites. Depending on the type of packing, the underlying crystal structure is referred to as an “end-on” or “interdigitating” crystal structure, as shown schematically in FIG. 1. In the case of the more densely packed “interdigitating” structure, the side chains protrude into the spaces between the side chains of the neighboring molecules and are thus parallel to the neighboring side chains. In contrast, the side chains in the “end-on” crystal structure are oriented frontally to the side chains of the neighboring molecules. The crystallite thickness (d) is therefore significantly smaller in the case of the “interdigitating” crystal structure than in the case of the “end-on” crystal structure. In the ideally packed case, the crystallite thickness for the “interdigitating” crystal structure (dᵢ) corresponds to the length of the side chain. The crystallite thickness in the ideally packed “end-on” crystal structure (dₑ) corresponds to twice the length of the side chain plus a C-C distance between the two ends of the frontally packed side chains.

If the above-defined at least one copolymer with the formula (I) has a melting temperature (Tₘ) of 20 to 35° C. and a melting enthalpy (ΔHₘ) of 20 J/g or more, the thermoresponsive wound dressing according to the invention has a reliable peel strength at 0 to 37° C. on the skin of the corresponding patient.

The peel strength of the thermoresponsive wound dressing according to the invention, comprising such adhesive compositions as described above, at 0 to 37° C. is 0.30 to 8.00 N/10 mm, preferably 0.40 to 7.50 N/10 mm, more preferably 0.60 to 6.00 N/10 mm, particularly preferably 0.80 to 5.50 N/10 mm. For example, the peel strength at 0 to 37° C. can be 0.30 to 7.50 N/10 mm, 0.30 to 6.00 N/10 mm, 0.30 to 5.50 N/10 mm, 0.40 to 8.00 N/10 mm, 0.40 to 6.00 N/10 mm, 0.40 to 5.50 N/10 mm, 0.60 to 8.00 N/10 mm, 0.60 to 7.50 N/10 mm, 0.60 to 5.50 N/10 mm, 0.80 to 8.00 N/10 mm, 0.80 to 7.50 N/10 mm or 0.80 to 6.00 N/10 mm.

The peel strength of the thermoresponsive wound dressing according to the invention can be determined at 0 to 37° C. using a 180° peel test according to “ASTM D3330 Test Method A 180 Degree Peel Test”.

If the above-defined at least one copolymer with the formula (I) has a melting temperature (Tₘ) of 20 to 35° C. and a melting enthalpy (ΔHₘ) of 20 J/g or more, the thermoresponsive wound dressing according to the invention enables the wound dressing to be removed from the patient's skin after cooling without causing pain and leaving residues.

In addition, the above-defined copolymer with the formula (I) has excellent biocompatibility. Specifically, any hydrolysis of the above-described copolymer with the formula (I) only produces polyvinyl alcohol (PVA) and fatty acids, which are considered medically harmless.

The biocompatibility of the copolymer according to formula (I) used according to the invention can be evaluated according to ISO 10993-05.

Preferably, the above-defined adhesive composition can further comprise at least one antimicrobial substance. Consequently, the above-defined adhesive composition can comprise one antimicrobial substance or two or more antimicrobial substances.

Here, antimicrobial substances are to be understood as chemical substances that reduce the reproductivity or infectivity of microorganisms, e.g. bacteria and viruses, or render them harmless or inactivate them. According to the invention, the antimicrobial substances are not restricted as long as they have an antimicrobial effect. Antimicrobial substances for wound dressings are already known and may be chlorhexidine, thymol, eugenol, chlorophenols, phosphoric acid salts containing silver ions, alginates, chlorodiphenyl ethers or natural or synthetic zeolites containing silver, copper or zinc.

Preferably, the above-defined adhesive composition further comprises at least one antimicrobial substance in an amount of 1 to 30 wt. %, more preferably 3 to 25 wt. %, particularly preferably 5 to 20 wt. %. For example, the above-defined adhesive composition may comprise at least one microbial substance in an amount of 1 to 25 wt. %, 1 to 20 wt. %, 3 to 30 wt. %, 3 to 20 wt. %, 5 to 30 wt. % or 5 to 25 wt. %.

The above-defined adhesive composition may further comprise tackifiers such as colophony or polyester, antioxidants such as vitamin E, fibrous or non-fibrous fillers, dyes or combinations thereof.

The above at least one copolymer with the formula (I) can be prepared, for example, by the following elimination reaction:

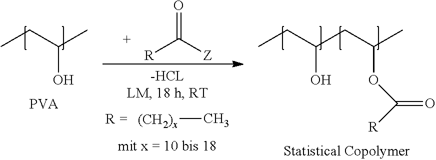

where LM stands for solvent and RT stands for room temperature. Examples of solvents that can be used are N-methyl-2-pyrrolidone (NMP), N-methylcaprolactam, N-vinyl-2-pyrrolidone, N-methylimidazole, N-methylimidazolium chloride, cresol, eugenol, ionic liquids or mixtures thereof. PVA can be, for example, polyvinyl alcohol, a fully or partially hydrolyzed polyvinyl acetate or a fully or partially hydrolyzed ethylene/vinyl acetate copolymer. However, the above-mentioned elimination reaction can also be carried out in a melt without solvent LM. Z can be, for example, fluorine, chlorine, bromine, iodine, phenolate, chlorophenolate, nitrophenolate, eugenolate and/or glycerin.

The ratio of vinyl alcohol units to vinyl fatty acid ester units of the above at least one copolymer with the formula (I) can be adjusted accordingly by varying the molar ratio [PVA]/[alkyl acid-R′]. After completion of the elimination reaction described above, the resulting copolymer can be further purified as a crude product by dissolving and precipitating once or several times in appropriately suitable solvents and precipitants as well as by extraction and dialysis.

Alternatively, the corresponding vinyl fatty acid carboxylate, for example vinyl myristic acid carboxylate, can be homopolymerized or copolymerized with vinyl acetate, which then results in fully esterified polyvinyl alcohols. Vinyl alcohol units can then be formed by transesterification with mono or polyfunctional alcohols.

If polyvinyl alcohol or partially hydrolyzed polyvinyl acetates or corresponding ethylene/vinyl acetate (EVA) copolymers that are either fully or partially hydrolyzed are used as a starting material with corresponding vinyl fatty acid carboxylate, for example methyl myristic acid ester, the lower-boiling by-product methyl acetate can advantageously be separated more easily, thus avoiding the handling of acid chlorides as reaction partners.

The carrier material of the thermoresponsive wound dressing according to the invention is not further restricted, provided that the above-defined adhesive composition can be applied thereto.

The carrier material of the thermoresponsive wound dressing according to the invention preferably consists of at least one material selected from the group consisting of cellulose, polyvinyl chloride, polyethylene, polyethylene terephthalate, polyurethane and polyether ester.

The thermoresponsive wound dressing according to the invention can be produced by applying an above-defined adhesive composition to one of the surfaces of the carrier element. The application is not further restricted, provided that the above adhesive composition is applied to one of the surfaces of the carrier element. The application of the above adhesive composition to one of the surfaces of the carrier element can be realized by coating, spraying, 3D printing, rolling or dipping, for example. Coating can be carried out with melts, solutions or dispersions of one or more of the above copolymers according to formula (I).

In addition to wound healing, the present invention also relates to the use of the thermoresponsive wound dressings according to the invention to protect against skin injuries and/or skin disorders. The use of the above-defined thermoresponsive wound dressings is not further restricted according to the invention as long as they protect against skin injuries and/or skin disorders.

Various skin injuries and/or skin disorders for which wound dressings are used are known to the person skilled in the art and may be burns, lacerations and cuts, for example.

The figures show:

FIG. 1 shows possible crystal structures of polymers through side chain crystallization: “interdigitating” crystal structure (left); “end-on” crystal structure (right).

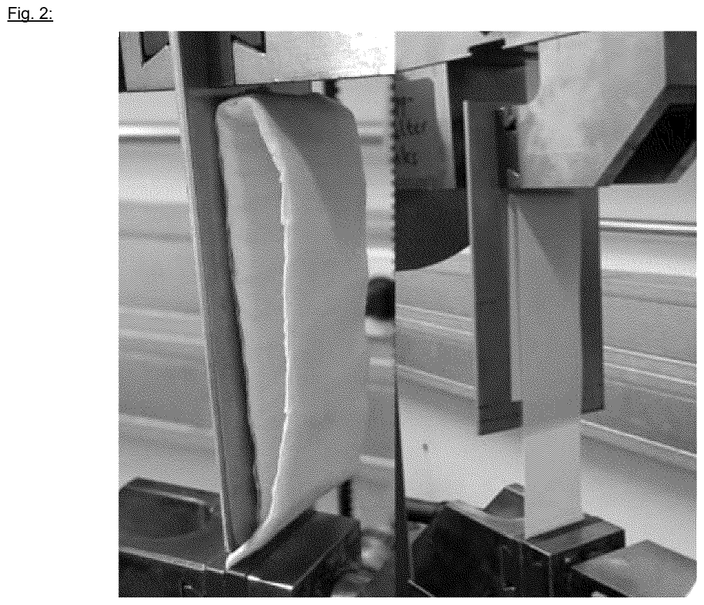

FIG. 2 shows the test setup for 180° peel tests according to “ASTM D3330 Test Method A 180 Degree Peel Test”: wound dressing according to the present invention (left); Leukoplast® reference (right).

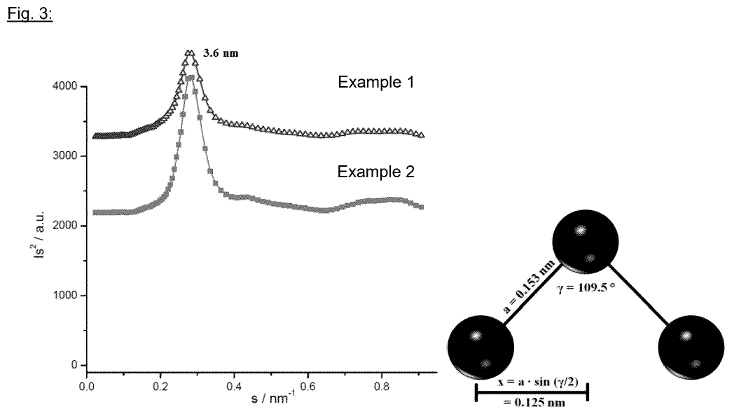

FIG. 3 shows on the left: SAXS diffractograms of examples 1 and 2; on the right: distance between two carbon atoms along an alkyl side chain assuming 0.153 nm bond length and tetrahedral angle.

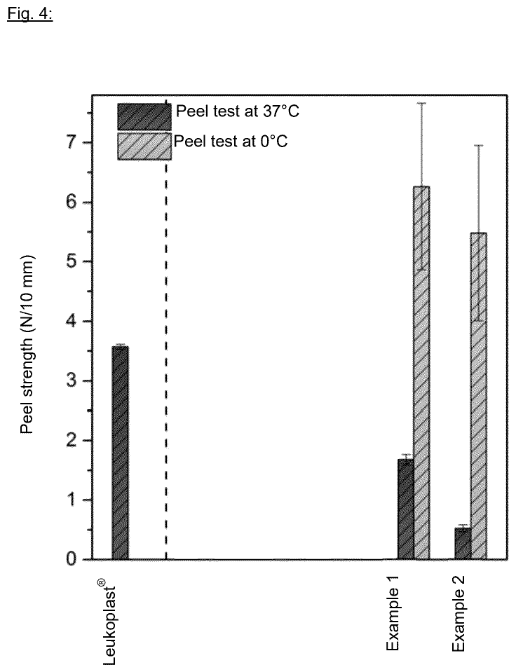

FIG. 4 shows the peel strength in N/10 mm of examples 1 and 2 at 37° C. and 0° C. in comparison to a Leukoplast® tape.

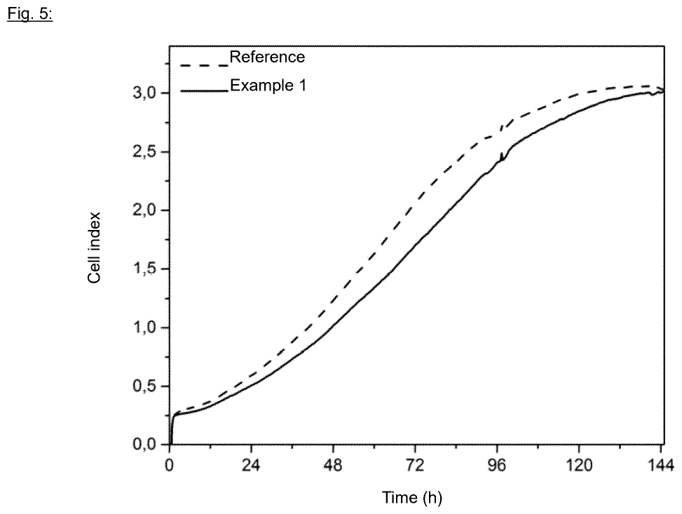

FIG. 5 shows the plot of the cell index against time as a measure of cell growth in the eluate test of Example 1 compared to a reference sample.

### EXAMPLES

The following examples serve to illustrate the present invention, but are not limited thereto.

### a) Materials and Chemicals Used:

Azo-bis-(isobutyronitrile) (AIBN, ≥98%), isopropanol (≥99.9%), vinyl stearate (≥ 99.9%, stabilized with mequinol), polyvinyl alcohol (PVA, Mowiol 6-98, 98% degree of hydrolysis, Mₙ=47000 g/mol), palmitoyl chloride (98%), myristoyl chloride (97%) and lauroyl chloride (98%) were from Sigma-Aldrich Chemie GmbH. Vinyl palmitate (96%, stabilized with mequinol) was obtained from abcr GmbH. Toluene (≥99.9%) and N-methyl-2-pyrollidone (NMP, ≥99.8%) were from Carl Roth GmbH & Co. KG. Ethanol (EtOH, ≥99.9%), chloroform (≥99.98%) and dimethyl sulfoxide (DMSO, ≥99.9%) were purchased from Fisher Chemical. Diethyl ether (≥99.9%) was from VWR. Deuterated chloroform (CDCl₃, ≥99.8%) was purchased from Eurisotop GmbH. Acetone (techn.) and methanol (MeOH, techn.) were from Oelfabrik Schmidt GmbH. The wound dressings used for sample preparation were Zemuko® nonwoven compresses (PZN: 01226396).

### b) Characterization Methods:

### DSC (Differential Scanning Calorimetry)

The melting temperature (Tₘ) and the melting enthalpy (ΔHₘ) of the statistical copolymers were determined using DSC with the aid of a “DSC 204 F1 Phoenix” from Netzsch Gerätebau GmbH. The samples were cooled from room temperature to −50° C., then heated to +100° C., cooled again to −50° C. and heated once more to +100° C. The heating rate was 10 K/min in all cases. Before the start of each heating or cooling phase, the sample was kept at the respective starting temperature for 5 minutes. The software “Netzsch Proteus 6.1” was used for the final evaluation.

### Small-Angle X-Ray Scattering (SAXS)

The SAXS experiments were carried out at room temperature with an evacuated Kratky compact camera with line focus (Anton Paar, Graz, Austria) and a position-sensitive detector (Hecus-mBraun, Graz, Austria). The X-ray source was an X-ray tube with a copper anode, wavelength λ=0.1542 nm, s=(2/A) sine. The Lorentz-corrected scattering curves were finally plotted graphically after the substrate had been removed.

### Peel Strength

To examine the peel strength of the thermoresponsive wound dressings, 180° peel tests were carried out in accordance with the standard “ASTM D3330 Test Method A 180 Degree Peel Test”. The measurement was carried out using a Zwick testing machine of type Z-005. The distance between the two clamping heads was 100 mm and the standard force was recorded with a 100 N load cell. The measuring speed was 5 mm/s. The “Zwick Test Xpert Software Version 11.0” was used for the final data evaluation.

For peel tests according to the “ASTM D3330 Test Method A 180 Degree Peel Test” standard, test specimens (dimensions: 200×30 mm) were cut from a Zemuko® wound dressing and evenly coated with 600 mg of melted adhesive composition on a defined adhesive surface (dimensions: 80×15 mm) using a spatula. The prepared wound dressings were annealed on a stainless steel plate (dimensions: 125×50×2 mm) while weighted down with a brass plate (m=2800 g) on a heatable aluminum block (dimensions: 15×15×3 cm) for 5 minutes at 37° C. For the tests at 0° C., the test specimens were then cooled with ice for 120 s. For the tests at 37° C., the samples were measured directly after annealing. To facilitate the reproduction of the 180° tensile tests, FIG. 2 shows the test setup using a prepared wound dressing and the Leukoplast® reference. The metal plate onto which the wound dressing was glued is clamped in the upper clamping head of the tensile/elongation machine. The end piece of the wound compress, which was folded over 180° above the adhesive surface, is clamped in the lower clamping head.

Residue of the Adhesive Composition after Removal of the Wound Dressings

For the peel tests, test specimens (dimensions: 60×30 mm) were cut from a Zemuko® wound dressing and evenly coated with 300 mg of melted adhesive composition on a defined surface (dimensions: 40×15 mm) using a spatula. The prepared wound dressings were annealed on a heatable aluminum block (dimensions: 15×15×3 cm) while loaded with a brass plate (m=2800 g) for 5 minutes at 37° C. The aluminum block was then placed on ice at 0° C. for 120 s. The wound dressings were then mechanically peeled off the aluminum block according to “ASTM D3330 Test Method A 180 Degree Peel Test” and the adhesive residue was determined by weighing the wound dressing before and after the peel test. 5 samples were examined for each adhesive composition. The error indicated corresponds to the standard deviation.

### Biocompatibility Test

To assess the biocompatibility of the thermoresponsive wound dressings, eluate tests were carried out to exclude the release of substances that inhibit cell growth. To do this, 200 mg of the samples to be tested were placed in an “E Plate 8 container” and 500 μL of cell culture medium (HPV 16 E6/E7-immortalized Keratinocyte Growth Medium 2 from Promocell) was added. The cells that were settled in the medium were keratinocytes from the oral mucosa (HaCaT cells from CLS Cell Lines Service). The impedance measurements, with which the cell index was calculated as a measure of cell growth, were carried out using the iCelligence device from ACEA Biosciences.

### c) Preparation of the Copolymers:

### Synthesis of Statistical Vinyl Alcohol/Vinyl Alkyl Ester Copolymers

To synthesize the statistical vinyl alcohol/vinyl alkyl ester copolymers, PVA (2.60 g, 59.0 mmol) was dissolved in NMP (40.0 mL) at 110° C. under a nitrogen atmosphere. The respective alkyl acid chloride was then added dropwise at RT. The reaction mixture was stirred at RT for 18 h before the crude product was precipitated by adding deionized water, filtered off and washed with water. The crude product was then purified by dissolving and precipitating several times in suitable solvents and precipitants until NMP could no longer be detected as a solvent in the ¹H-NMR spectrum (CDCl₃). Subsequently, the products obtained were dried in a vacuum drying cabinet at 60° C. for 18 h. Table 1 lists a summary of the amounts of reactants used and the solvents and precipitants used for the purification of two exemplary approaches.

TABLE 1

| | | Myristoyl | | |
|---|---|---|---|---|
| | PVA | chloride | | |
| Example | (mmol) | (mmol) | Solvent | Precipitant |
| Example 1 | 59.0 | 44.4 | CHCl₃ | MeOH |
| Example 2 | 59.0 | 59.2 | CHCl₃ | MeOH |

### d) Properties of the Copolymers:

Melting Temperature (Tₘ) and melting enthalpy (ΔHₘ)

The statistical copolymers obtained from Examples 1 and 2 were then examined with regard to a) the actual degree of esterification in formula (I), b) melting temperature (Tₘ) and c) melting enthalpy (ΔHₘ). The results obtained are summarized in Table 2.

TABLE 2

| | | Ratio of vinyl | | |
|---|---|---|---|---|
| | | alcohol units/vinyl | | |
| | x | fatty acid ester | | |
| Example | (Formula (I)) | units (Formula (I)) | Tₘ (° C.) | ΔHₘ (J/g) |
| Example | 12 | 0.38 | 26-29 | 35.9 |
| 1 | | | | |
| Example | 12 | 0.06 | 22-25 | 40.6 |
| 2 | | | | |

The DSC measurements of the statistical vinyl alcohol/vinyl alkyl ester copolymers produced showed that the melting behavior of the materials can be controlled by the composition. The starting material PVA melts at 217° C. The esterification of the alcohol groups of the PVA completely prevents the crystallization of the PVA polymer backbone and the influence of the side chain length and the side chain concentration dominate the melting behavior. The inventors found that the higher the degree of esterification and the longer the side chain, the greater the melting enthalpy (ΔHₘ). The above examples 1 and 2 meet all the required conditions for the thermal properties of a “bond/debond-on-command” material and thus for use as adhesive compositions for thermoresponsive wound dressings.

### Side Chain Crystallization

The results of the thermal properties of the statistical copolymers from the DSC measurements showed a strong influence on the melting behavior due to the length and concentration of the various fatty acid side chains. This is consistent with the assumption that the melting and crystallization of the material is caused by side chain crystallization.

To further support this hypothesis, X-ray diffraction experiments were carried out. In the SAXS (small angle x-ray scattering) experiment, superstructures of crystallites in the material can be determined. In the case of side chain crystallization, there are two different crystallization mechanisms by which the side chains can accumulate to form crystallites. Depending on the type of packing, the underlying crystal structure is referred to as an “end-on” or “interdigitating” crystal structure, as shown schematically in FIG. 1.

In the case of the more densely packed “interdigitating” crystal structure, the side chains protrude into the spaces between the side chains of the neighboring molecules and are thus parallel to the neighboring side chains. In contrast, the side chains in the “end-on” crystal structure are oriented frontally to the side chains of the neighboring molecules. The crystallite thickness (d) is therefore significantly smaller in the case of the “interdigitating” crystal structure than in the case of the “end-on” crystal structure. In the ideally packed case, the crystallite thickness for the “interdigitating” crystal structure (dᵢ) corresponds to the length of the side chain. The crystallite thickness in the ideally packed “end-on” crystal structure (dₑ) corresponds to twice the length of the side chain plus a C-C distance between the two ends of the frontally packed side chains.

The examination of the underlying crystallization mechanism was carried out using SAXS measurements based on Examples 1 and 2. FIG. 3 shows the results of the SAXS experiments carried out.

The distances determined from the SAXS diffractogram are 3.6 nm in the case of Examples 1 and 2. This speaks against the presence of an “interdigitating” crystal structure and is a strong indication that the materials investigated crystallize preferably in the “end-on” mechanism. In the case of a bond length of 0.153 nm and a tetrahedral angle of 109.5°, the distance between two C atoms along the alkyl side chain is 0.125 nm. Table 3 compares the ideal crystallite thicknesses dᵢ and dₑ calculated from this with the measured lattice constants from the diffraction experiment.

It can be clearly seen that the measured distances (d_{measured}) are too large for the presence of an “interdigitating” crystal structure, while the values are very close to the theoretically calculated values for an ideal “end-on” crystallization. One possible explanation for this is that there is little space between the side chains due to the high degree of esterification of the samples examined. Thus, the side chains of neighboring molecules cannot become incorporated into the spaces, but rather orient themselves frontally toward the fatty acid residues of the neighboring polymer chains.

TABLE 3

| Example | dᵢ, ideal (nm) | dₑ, ideal (nm) | d_{measured} (nm) |
|---|---|---|---|
| Example 1 | 1.875 | 3.875 | 3.900 |
| Example 2 | 1.875 | 3.875 | 3.900 |

Residue of the Adhesive Composition after Removal of the Wound Dressings

In addition to melting the statistical copolymer by body heat (approx. 37° C.), a crystallization of the alkyl ester side chains of the statistical copolymer is initiated using a commercially available cold pack or ice pack (approx. 0° C.). This greatly increases the cohesion of the adhesive composition and it peels off the patient's skin largely without any residues.

For this purpose, sample bodies were cut out of a commercially available Zemuko® wound dressing (dimensions: 6.0×3.0 cm) and a defined adhesive surface (4.0×1.5 cm) was evenly coated with melted statistical copolymer (300 mg each). The samples were each annealed for 5 minutes at 37° C. while weighted down with a brass plate (m=2800 g) and then cooled to 0° C. so that peel tests could then be carried out. Table 4 shows the results of the peel tests on aluminum. The adhesive residue in mass % was determined by the weight of the samples before and after peeling.

TABLE 4

| | | Adhesive residue |
|---|---|---|
| | | on aluminum |
| Example | Cooling time (s) | (mass-%) |
| Example 1 | 120 | 0.00 ± 0.10 |
| Example 2 | 120 | 0.00 ± 0.10 |

In this context, corresponding peel tests were used to determine that the adhesive composition itself has a strong influence on the amount of adhesive residue in the peel tests on aluminum. The higher the proportion of long-chain alkyl ester side chains, the lower the adhesive residue on the surfaces examined. As a comparative example, the adhesive residue of a Leukoplast® tape at 37° C. on aluminum is 0.40±0.13 mass %.

The explanation for this trend lies in the side chain crystallization. The higher the proportion of alkyl esters in the adhesive composition, the better the material can crystallize. The crystallites act as physical cross-linking points and thereby increase the cohesion of the material, which reduces the adhesive residue. An extension of the cooling time beyond 120 s was not carried out in order to take into account the patient's well-being during later use.

### Peel Strength

In order to be able to quantify the stickiness of the produced Examples 1 and 2, peel tests were carried out at a peel angle of 180° according to “ASTM D3330 Test Method A 180 Degree Peel Test”. The peel force is measured during peeling using a tensile/elongation machine. After a short start-up phase, it changes to a plateau and is indicated as the peel force in N/10 mm.

In order to be able to classify the results better, commercially available Leukoplast® tape was measured as a reference. The results in the previous section showed that with a cooling time of 120 s, the adhesive residue on aluminum is very small after the wound dressings have been removed. Therefore, all 180° peel tests were carried out by peeling the wound dressings off stainless steel plates after annealing for 5 min at 37° C. and after cooling for 120 s at 0° C.

The dimensions of the samples (commercially available Zemuko® wound compresses) were 200×30 mm each with an adhesive surface of 80×15 mm, which were evenly coated with 600 mg of melted adhesive composition. During annealing at 37° C., the samples were weighted down with a weight of 2800 g to simulate the pressing of the compresses onto the wound by the patient's hand. The resulting thermoresponsive peel strengths of Examples 1 and 2 from the peel tests at 37° C. and 0° C. compared to commercially available Leukoplast® tape at 37° C. are shown in FIG. 4.

At 37° C., a lower peel force is required to remove the wound dressings from the stainless steel plate in Examples 1 and 2 than in Leukoplast®. In particular, the peel strength for Examples 1 and 2 is approximately 15 to 47% compared to the reference Leukoplast®. This makes these adhesive compositions suitable for use as adhesive compositions for thermoresponsive wound dressings at 37° C.

In addition to reliable adhesion at body temperature, residue-free removal with the lowest possible peeling force is also important. The peeling tests at 0° C. show a strong influence of the adhesive composition on the peel strength. The higher the proportion of vinyl alkyl ester in the statistical copolymer, the lower the peeling force required. It was shown that in Examples 1 and 2, pain-free removal at 0° C. is possible.

### Biocompatibility

Since the statistical vinyl alcohol/vinyl alkyl ester copolymers produced are to be used as thermoresponsive adhesives on human skin, the biocompatibility of the materials used plays a major role. To ensure that no toxic components diffuse out of the material during use, eluate tests were carried out to evaluate biocompatibility in accordance with ISO 10993-05.

For this purpose, the statistical vinyl alcohol/vinyl alkyl ester copolymers were placed in cell culture medium for seven days at 37° C. and then cell cultivation was carried out using the medium in which the samples had previously been placed. These eluate tests were carried out using HaCaT cells from the company “CLS Cell Lines Service”. Cell growth was monitored over several days using impedance measurements and evaluated in the form of the “cell index”. By comparing with untreated cell culture medium as a reference, it can be concluded whether cell growth-inhibiting substances are migrating out of the material. In FIG. 5, the cell index is plotted against time for Example 1 and the reference. The almost identical curves of the respective sample with the reference in FIG. 5 show that cell growth was hardly affected by contact with Example 1. It was thus possible to ensure that no cell growth-inhibiting substances diffused from the materials into the medium over a period of seven days. This result represents good biocompatibility of the copolymers, which is a basic requirement for application on human skin.

1. A thermoresponsive wound dressing with thermally switchable adhesion/detachment, comprising:
a carrier material, and
an adhesive composition applied to a surface of the carrier material, the adhesive composition comprising:
at least one vinyl alcohol/copolymer with the following formula (I): 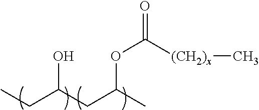
where x is an integer from 10 to 18 independently of one another with respect to each fatty acid unit,
where the copolymer according to formula (I) is present at body temperature as a melt that adheres to wounds and skin, and upon cooling to a temperature above the freezing point of water, the crystallization of the copolymer according to formula (I) causes the wound dressing to detach.

2. The thermoresponsive wound dressing according to claim 1, wherein the at least one copolymer according to formula (I) has a melting temperature (Tₘ) of 20 to 35° C. and a melting enthalpy (ΔHₘ) of 20 J/g or more.

3. The thermoresponsive wound dressing according to claim 1 or 2, wherein the at least one copolymer according to formula (I) has a number-average molar mass (Mₙ) of 1000 to 100,000 g/mol, wherein Mₙ is determined by gel permeation chromatography (GPC).

4. The thermoresponsive wound dressing according to one of claims 1 to 3, wherein the at least one copolymer according to formula (I) has a polydispersity index (PDI) of 1.0 to 3.0, wherein the PDI is determined by gel permeation chromatography (GPC).

5. The thermoresponsive wound dressing according to one of claims 1 to 4, wherein the adhesive composition comprises the at least one copolymer according to formula (I) in an amount in the range from 40 to 100 wt. %.

6. The thermoresponsive wound dressing according to one of claims 1 to 5, wherein the adhesive composition further comprises at least one antimicrobial substance.

7. The thermoresponsive wound dressing according to one of claims 1 to 6, wherein x is 12 and a molar ratio of vinyl alcohol units to vinyl fatty acid ester units of the at least one copolymer according to formula (I) is 0.05 to 0.50, preferably 0.06 to 0.40, determined by means of ¹H-NMR spectroscopy.

8. The thermoresponsive wound dressing according to one of claims 1 to 7, wherein the at least one copolymer according to formula (I) is a statistical copolymer.

9. The thermoresponsive wound dressing according to one of claims 1 to 8, wherein the at least one copolymer according to formula (I) further comprises one or more units derived from monomers selected from ethylene and vinyl acetate.

10. The thermoresponsive wound dressing according to one of claims 1 to 9, wherein the carrier material consists of at least one material selected from the group consisting of cellulose, polyvinyl chloride, polyethylene, polyethylene terephthalate, polyurethane and polyether ester.

11. A use of the thermoresponsive wound dressing according to one of claims 1 to 10 for wound healing or for protection against skin injuries and/or skin disorders.

12. A method for producing a thermoresponsive wound dressing according to one of claims 1 to 10, wherein the carrier material is coated with a melt, solution or dispersion of at least one copolymer according to formula (I), as defined above.
